(19) United States
(12) Patent Application Publication
(10) Pub. No.: US 2004/0087924 A1
(43) Pub. Date: May 6, 2004

(54) Semi-hydrophobic cover for an absorbent product

(75) Inventors: Michelle May Sroda, Green Bay, WI (US); Brian D. Babcock, Appleton, WI (US); Patricia A. Mitchler, Neenah, WI (US); Heather Ann Sorebo, Appleton, WI (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/444,865

(22) Filed: May 22, 2003

Related U.S. Application Data
(60) Provisional application No. 60/424,292, filed on Nov. 6, 2002.

(51) Int. Cl. A61F 13/15; A61F 13/20

(52) U.S. Cl. 604/367; 604/385.01

(57) ABSTRACT

An absorbent feminine care article (40) can include a liquid-permeable cover (62); and an absorbent member (66) which is operatively joined with the cover (62). The cover (62) can include a hydrophilic first layer portion (68) operatively joined with a relatively less hydrophilic, second layer portion (30). The second layer portion (30) can be positioned on a bodyside of the article (20); and the first layer portion (28) can be positioned between the second layer portion (30) and the absorbent member (66).

### FIELD

The present invention relates generally to an absorbent article. More particularly, the present invention relates to an absorbent, feminine care article, such as a feminine care pad.

### BACKGROUND

A broad manner and wide variety of absorbent articles configured for the absorption of bodily exudates such as menstrual fluid are well known. With respect to feminine hygiene, the art has offered two basic types of feminine hygiene protection: sanitary napkins, developed for external wear about the pudendal region, and tampons, developed for residence within the vaginal cavity and interruption of menstrual flow therefrom. Hybrid feminine hygiene protection devices, attempting to merge the structural features of both within a single type of device, have also been proposed, but have not seen a meaningful measure of acceptance. The ability to realize appropriate advantages has been overshadowed by the more demonstrable perpetuation of structural and functional disadvantages. Other less intrusive devices have also been proposed. Such devices have been known as labial or interlabial devices, and characterized as having a portion which at least partially resides external of the wearer's vestibule. Other, even smaller devices that may be worn interlabially by a female wearer, have also been produced. Additionally, there have been arrangements which combine the use of labial or interlabial devices with the use of other absorbent, feminine hygiene articles.

Conventional feminine hygiene systems, such as those described above, have not provided desired levels of comfort and/or absorbency, particularly for light flow protection. As a result, there has been a continued need for feminine care articles that can more rapidly intake liquids while providing greater discreteness and lower leakage.

### BRIEF DESCRIPTION

The present inventors have recognized the deficiencies and problems inherent in the prior art, and in response, have conducted intensive research in developing innovative absorbent, feminine care articles, such as absorbent labial pads. Generally stated, an absorbent feminine care article can include a liquid-permeable cover; and an absorbent member which is operatively joined with the cover. The cover can include a hydrophilic first layer portion operatively joined with a relatively less hydrophilic, second layer portion. In particular aspects, the second layer portion can be positioned on a bodyside of the article; and the first layer portion can be positioned between the second layer portion and the absorbent member.

In other aspects, the cover can include a hydrophilic first nonwoven layer operatively joined with a relatively less hydrophilic, second nonwoven layer. In further aspects, the first and second nonwoven layers can be laminated with a spunlace process, and can be selectively apertured.

By incorporating its various aspects and features, the article of the invention can better maintain a desired combination of rapid intake of liquid, low leakage and low irritation. As a result, the invention can help to increase the absorbent performance of the article, and help provide greater convenience and comfort to the user.

### DRAWINGS

The foregoing and other features, aspects, configurations and advantages of the present invention will become better understood with regard to the following description, claims and accompanying drawings where:

FIG. 1 shows a partially cut-away, top plan view of a representative feminine care article of the invention.

FIG. 1A shows a view of a representative cross-section through the feminine care article illustrated in FIG. 1.

FIG. 2 representatively shows a simplified anatomical cross-sectional view of a human female illustrating the external genitalia.

FIG. 2A representatively shows a simplified anatomical cross-sectional view of a human female illustrating the environment for a feminine care article, such as provided by a labial pad.

FIG. 3 representatively shows a simplified view illustrating the positioning of an inserted feminine care article having a notch located on that portion of the periphery thereof that is intended to be situated nearest the clitoris of a human female.

FIG. 4 representatively shows a top view illustrating a version of a feminine care article arranged in a generally flat condition.

FIG. 5 representatively shows cross-sectional view of the feminine care article illustrated in FIG. 4 taken along line 5-5 thereof.

FIG. 6 representatively shows a cross-sectional view illustrating another version of a feminine care article having a cover layer, a backsheet layer and a pliable component or member sandwiched between the cover and backsheet layers.

FIG. 7 representatively shows a top view illustrating an embodiment of a notch situated on the periphery of a feminine care article which is arranged in a generally flat condition.

FIG. 8 representatively shows a top view illustrating another embodiment of a notch situated on the periphery of yet another version of a feminine care article which is arranged in a generally flat condition and.

FIG. 9 representatively shows a top view illustrating yet another embodiment having a plurality of notches situated on the periphery of still another version, of a feminine care article which is arranged in a generally flat condition and.

FIG. 10 representatively shows a top view illustrating still another embodiment having a plurality of notches situated on the periphery of a further version of a feminine care article which is arranged in a generally flat condition and.

FIG. 11 representatively shows a cross-sectional view illustrating yet a further version of a feminine care article.

FIG. 12 representatively shows cross-sectional view illustrating the version of FIG. 12 in a selected, substantially folded position.

FIG. 13 illustrates a representative, enlarged view of another version of a feminine care article folded substantially about a principal axis.

FIG. 14 illustrates a representative, exaggerated enlarged view of an article folded substantially about a principal axis and being grasped for disposition in the vestibule by the wearer's fingers.

FIG. 15 shows a feminine care article which is arranged in a generally flat condition and has a representative system of one or more tab regions.

FIG. 16 shows a representative cross-section through a feminine care article having one or more tab regions.

FIG. 17 shows a graph which representatively compares the improved absorbency provided by an article which incorporates the present invention.

### DETAILED DESCRIPTION

It should also be noted that, when employed in the present disclosure, the terms “comprises”, “comprising” and other derivatives from the root term “comprise” are intended to be open-ended terms that specify the presence of any stated features, elements, integers, steps, or components, and are not intended to preclude the presence or addition of one or more other features, elements, integers, steps, components, or groups thereof. It should also be noted that in each of the Figures of the drawings, similar parts are identified with like reference characters.

“Disposable” includes being disposed of after a single use and not intended to be washed and reused.

“Hydrophilic” describes fibers or the surfaces of fibers that are wetted by the aqueous liquids in contact with the fibers. The degree of wetting of the materials can, in turn, be described in terms of the contact angles and the surface tensions of the liquids and materials involved. Equipment and techniques suitable for measuring the wettability of particular fiber materials can be provided by a Cahn SFA-222 Surface Force Analyzer System, or a substantially equivalent system. When measured with this system, fibers having contact angles less than 90° are designated “wettable” or hydrophilic, while fibers having contact angles equal to or greater than to 90° are designated “nonwettable” or hydrophobic. When comparing materials, a material that forms a relatively larger contact angle with water is relatively less hydrophilic than a material that forms a smaller contact angle with water.

As used herein the term “nonwoven fabric or web” means a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. No. 3,849,241 to Butin et al. Meltblown fibers are microfibers which may be continuous or discontinuous, are generally smaller than 10 microns in average diameter, and are generally tacky when deposited onto a collecting surface.

“Spunbonded fibers” refers to small diameter fibers that are formed by extruding molten thermoplastic material as filaments from a plurality of fine capillaries of a spinneret. Such a process is disclosed in, for example, U.S. Pat. No. 4,340,563 to Appel et al. and U.S. Pat. No. 3,802,817 to Matsuki et al. The fibers may also have shapes such as those described, for example, in U.S. Pat. No. 5,277,976 to Hogle et al. which describes fibers with unconventional shapes.

As used herein “hydroentangling” means a process wherein a nonwoven web, or layers of a non-woven web, are subjected to streams of a non-compressible fluid, e.g., water, at a high enough energy level and for a sufficient time to entangle the fibers thereof. The fluid may advantageously be used at a pressure of between about 200 and 5000 psig (14-351 kg/cm² gauge) from a distance of a few inches (centimeters) above the web while the web is supported by a mesh structure. This process is described in detail in U.S. Pat. No. 3,486,168 to Evans et al., which is incorporated herein by reference. Nonwoven webs subjected to hydroentangling are, for example, referred to as “spunlace” fabrics.

“Bonded carded web” refers to webs that are made from staple fibers which are sent through a combing or carding unit, which separates or breaks apart and aligns the staple fibers in the machine direction to form a generally machine direction-oriented fibrous nonwoven web. This material may be bonded together by methods that include point bonding, through air bonding, ultrasonic bonding, adhesive bonding, etc.

“Airlaying” is a well-known process by which a fibrous nonwoven layer can be formed. In the airlaying process, bundles of small fibers having typical lengths ranging from about 3 to about 52 millimeters (mm) are separated and entrained in an air supply and then deposited onto a forming screen, usually with the assistance of a vacuum supply. The randomly deposited fibers then are bonded to one another using, for example, hot air or a spray adhesive. Airlaying is taught in, for example, U.S. Pat. No. 4,640,810 to Laursen et al.

“Feminine care article” means products such as sanitary napkins, pantiliners, tampons, interlabial devices, miniforms and the like which have been operatively configured for the absorption of body exudates, such as urine, menses, vaginal fluids and the like.

“Target area” refers to the area or position on a personal care product where an insult is normally delivered by a wearer.

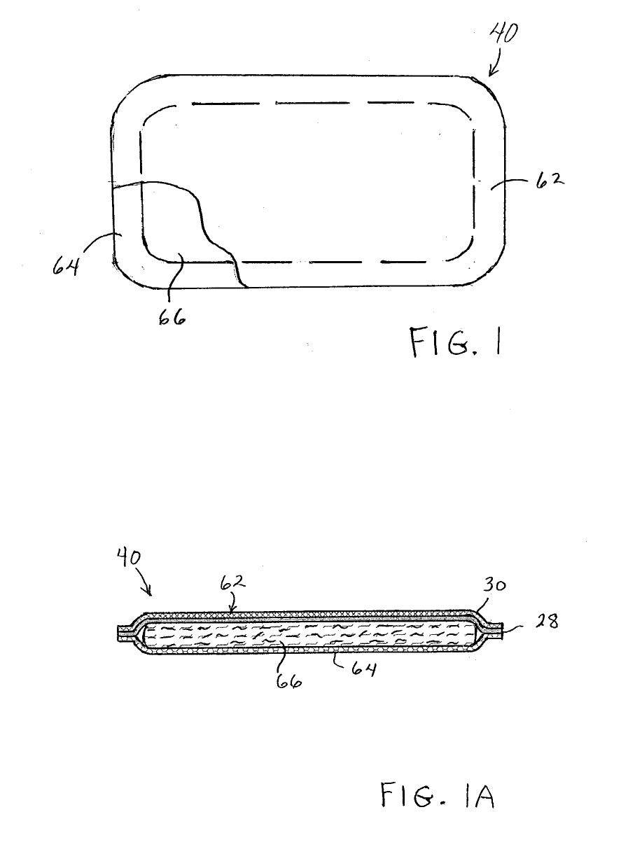

With reference to FIGS. 1 and 1A, an absorbent feminine care article 40 can include a liquid-permeable cover 62; and an absorbent member 66 which is operatively joined with the cover 62. The cover 62 can include a hydrophilic first layer portion 68 operatively joined with a relatively less hydrophilic, second layer portion 30. In a particular aspect, the second layer portion 30 can be positioned on a bodyside of the article 20; and the first layer portion 28 can be positioned between the second layer portion 30 and the absorbent member 66.

In another aspect, the cover 62 can comprise a unitary nonwoven fabric having a first, major facing-surface portion; and a major, second facing-surface portion which is positioned opposite the first, major facing-surface portion. The hydrophilic, first layer portion 28 of the cover can be provided by the first facing-surface portion; and the relatively less-hydrophilic, second layer portion 30 of the cover can be provided by the second facing-surface portion. Additionally, the hydrophilic, first facing-surface portion of the cover can include a surfactant.

In a further aspect, the cover 62 can include a hydrophilic first nonwoven stratum or layer 28 which can be laminated or otherwise operatively joined with a relatively less hydrophilic, second nonwoven stratum or layer 30. For example, the nonwoven layers of the cover can be joined with a hydroentangling, spunlace operation. The second nonwoven stratum or layer 30 of the cover can be positioned on a bodyside of the article 20; and the first nonwoven stratum or layer 28 of the cover can be positioned between the second nonwoven layer 30 and the absorbent member 66. In particular aspects, either or both of the first and second nonwoven layers 28, 30 can be apertured. An additional aspect of the cover may include a second layer portion 30 that has been configured to be substantially hydrophobic. In a desired feature, the feminine care article can be configured to provide a disposable absorbent article.

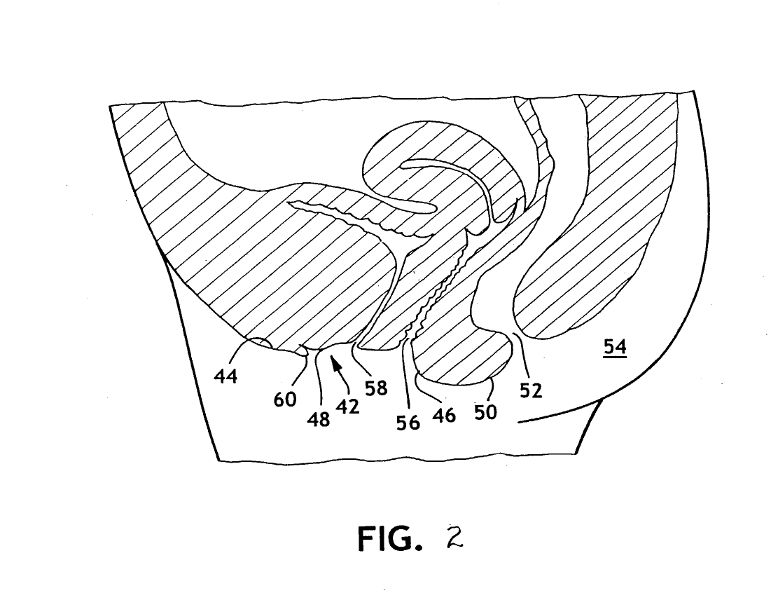

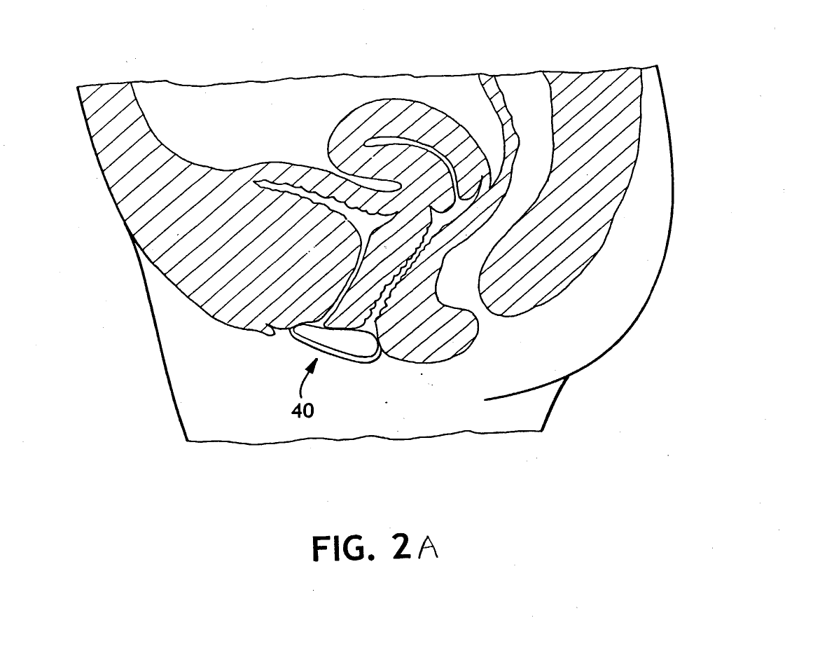

With reference to FIGS. 2 and 2A, the vulva refers generally to the external female genitalia, including the labia minora, labia majora, clitoris 60 and vestibule 42. The vestibule 42 is considered to be the region defined within the labia beginning at about a point lying caudally from the anterior labial commissure 44, extending rearward to the posterior labial commissure 46 and bounded inwardly by the floor 48 of the vestibule. One of skill in the art fully understands that there is a wide range of variation among women with respect to the relative size and shape of labia minora and labia majora as the same interrelatedly define the contour of the vestibule 42. For purposes of the present description, however, such differences will not specifically be addressed, it being recognized that in any event the disposition of the feminine care article into the vestibule 42 will necessitate placement between the labia majora regardless of any such consideration respecting the labia minora. Lying caudally of the vestibule 42 is the perineum 50 which leads to the anus 52 in the region of the buttocks 54. Within the vestibule 42 itself is located the principal urogenital members which, for purposes pertinent here, are constituted of the vaginal orifice 56, the urethral orifice 58, and the clitoris 60. Given the foregoing simplified review of this anatomical region, and to facilitate the present description, the vestibule 42 will be considered generally to be the region between the posterior labial commissure 46 and the clitoris 60, for convenience. For a more comprehensive description of this portion of the human female anatomy, however, reference can be made to Anatomy of the Human Body by Henry Gray, Thirtieth American Edition Carmine D. Clemente ed., Lea & Febiger, 1985 at 1571-1581.

FIG. 3 diagrammatically illustrates a representative feminine care article, such as provided by a labial pad or other interlabial device 40, which is shown in a substantially unfolded or flat configuration prior to disposition within the vestibule of a wearer. The interlabial device may optionally include at least one absorbent component. Additionally, the interlabial device can be configured for disposition in between the labia majora, and can be configured to extend at least partially into the vestibule 42 of a female wearer during use.

The feminine care article, such as provided by the labial pad, miniform or other interlabial device 40, can be disposed at least partially within the vestibule 42 to operatively occlude the vestibule region and to operatively block or otherwise operatively restrict an undesired movement of material from the vulva-vaginal region of a female user. Desirably, the interlabial device or other feminine care article can be disposed substantially entirely within the vestibule region. The feminine care article may optionally be configured to provide an absorbent article which can be employed to provide for a desired intake and retention of a selected liquid, such as menstrual fluid or other liquid emitted from the vaginal orifice 56. Additionally, the feminine care article may optionally serve as a type of incontinence device for the absorption and storage of urine, as may be desired to address minor, female incontinence. In a particular arrangement, the feminine care article can be configured to operatively impede an undesired movement of a selected treatment material from the vulva-vaginal area, when the interlabial device is placed in the interlabial space of the user/wearer. In a another feature, the feminine care article can be configured to be operatively occlusive and substantially nonabsorbent at least with respect to the selected treatment, and may be operatively permeable and absorbent with respect to ordinary bodily fluids, such as urine, vaginal fluids or the like. Where the feminine care article comprises an interlabial device, the article may include additional components. For example, the interlabial device may be attached to or otherwise configured or employed in combination with another feminine care article, such as feminine care absorbent pad that is employed to retain urine, menses and/or other vaginal fluid.

Examples of known interlabial devices are described in U.S. Pat. No. 4,595,392 entitled INTERLABIAL PAD by Russell L. Johnson et al. which was issued Jun. 17, 1986; and in U.S. Pat. No. 4,743,245 entitled LABIAL SANITARY PAD by Frederich O. Lassen et al. which was issued May 10, 1988. The entire disclosures of these documents are incorporated herein by reference in a manner that is consistent herewith.

Interlabial pads are also described in U.S. patent application Ser. No. 10/036,990 entitled LABIAL PAD HAVING A NOTCH by James J. Hlaban et al. which was filed Dec. 31, 2001 (attorney docket No. 17,694); and in U.S. patent application Ser. No. 10/036,635 entitled LABIAL PAD HAVING A NOTCH by Ronald L. Edens et al. which was filed Dec. 31, 2001 (attorney docket No. 17,698). The entire disclosures of these documents are incorporated herein by reference in a manner that is consistent herewith.

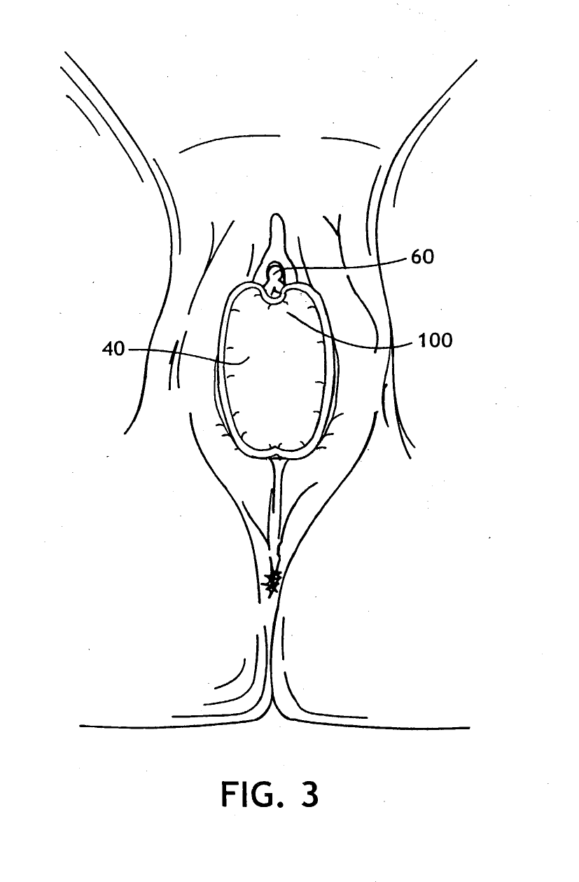

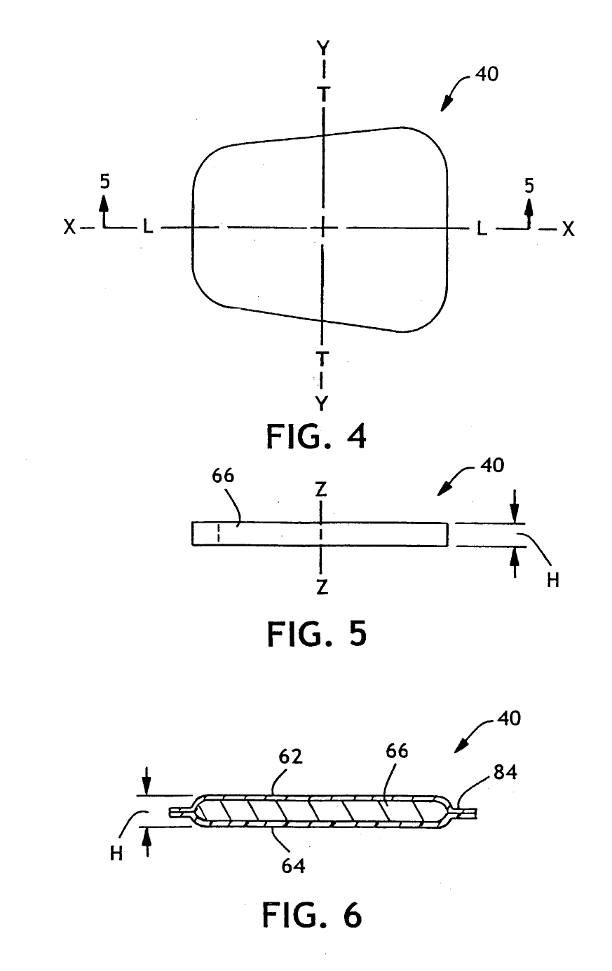

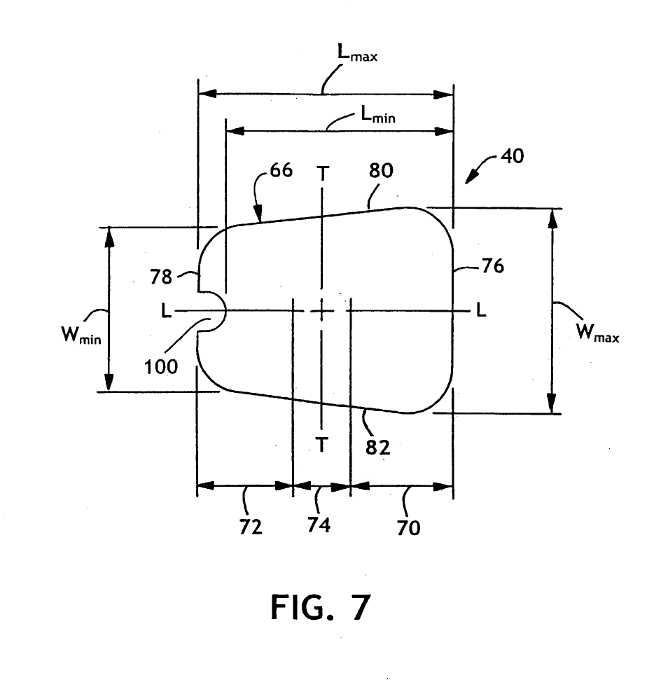

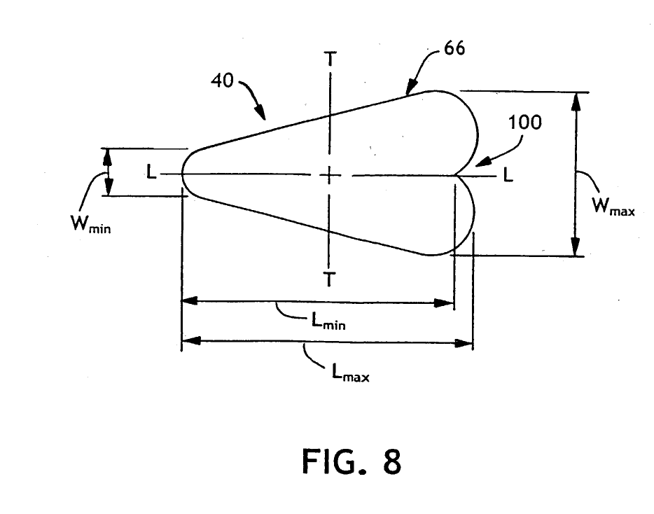

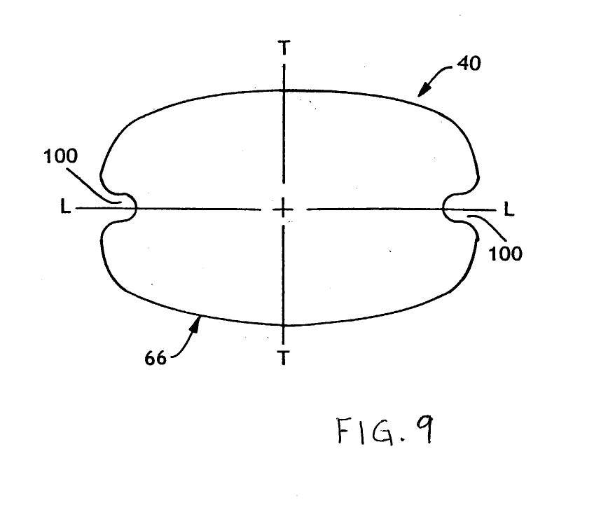

As illustrated in FIG. 4, the representative feminine care article (e.g. the interlabial device 40) can have a principal longitudinal axis (L) which generally runs along the x-direction. As used herein, the term “longitudinal” refers to a line, axis or direction which lies in the general plane of the feminine care article, and when the feminine care article is in use, is generally aligned with (e.g., approximately parallel to) a vertical plane that bisects a standing female wearer into left and right body halves. The longitudinal direction is generally illustrated in FIG. 4 by the x-axis. The feminine care article also has a principal transverse axis (T). The terms “transverse,” “lateral” or “y-direction” as used herein generally refer to a line, axis or direction that is generally perpendicular to the longitudinal direction. The lateral direction is generally illustrated in FIG. 4 by the y-axis, and typically lies generally parallel to a representative plane of the article. The “z-direction” is typically a line, axis or direction generally parallel to the vertical plane described above, and is generally perpendicular to both the longitudinal (x-axis) and transverse (y-axis) directions. The z-direction is generally illustrated in FIG. 5 by the z-axis. The term “upper” refers generally to an orientation directed toward the wearer's head, while the terms “lower” or “downwardly” refer generally to an orientation directed toward the wearer's feet. For purposes of discussion herein, each layer of the feminine care article, e.g., a cover 62, a backsheet or baffle 64 and/or a pliable member 66 (e.g. FIG. 6), has an upper or body-facing surface and a lower surface also described as the surface opposed to the upper or body-facing surface. The pliable component 66 can be sufficiently flexed, molded, folded and/or shaped to provide a desired resiliency or other pliability which operatively allows a selective configuring of the feminine care article for its intended use by an individual person. In a particular aspect, the pliable component can be selectively shaped for interlabial placement in the vestibule region of the wearer, and can be selectively reconfigured by the individual wearer to provide a customized placement and fit of the feminine care article in the interlabial space of the wearer. As a result, the contours of the pliable member and the feminine care article can be tailored to more effectively match the body contours of the individual wearer, and can be customized to provide a tailored fit that can more effectively maintain the desired location of the selected treatment material. The pliable component 66 may optionally have a selected level of absorbent capacity, and may be configured to provide an operative, absorbent body or absorbent core.

As illustrated in FIG. 6, a representative article (e.g. the illustrated interlabial device 40) can include a liquid-permeable cover or topsheet layer 62, a baffle or backsheet layer 64 which may be operatively liquid-impermeable, and a pliable component member 66 which is operatively situated between the cover and the baffle. The pliable component 66 may optionally be configured as an absorbent body or absorbent core, and may have a composite structure. As illustrated in FIG. 7, the pliable component 66 and the article can each have a first end region 70, a second end region 72, and a central region 74 disposed between the first and second end regions 70, 72, respectively. The feminine care article has a suitable size and shape that allow at least a portion of the feminine care article to be disposed within the vestibule 42 of a female wearer. In addition, the article can at least partially occlude and intercept the flow of a selected treatment material, menstrual fluid, urine or other bodily exudate from the wearer's vaginal orifice 56 and/or urethral orifice 58.

The pliable component 66, and thus the feminine care article, can generally display a geometry extending between spaced apart first 76 and second 78 transverse end areas. The overall geometry is completed by noting that the pliable component 66, and thus the feminine care article, also includes spaced apart first 80 and second 82 longitudinal sides ranging between the transverse end areas 76, 78, these collectively sometimes being referred to herein as the perimetric sides i.e., those defining the periphery.

The geometry of the pliable component 66 can be a significant factor affecting the overall size and effectiveness of the feminine care article (e.g. the interlabial device 40). In general, the pliable component 66 can have a maximum width (Wₘₐₓ), measured along a line laying generally parallel to the principal transverse axis (T) and running from one longitudinal side 80 to the opposing longitudinal side 82, and a minimum width (Wₘᵢₙ) measured along a second line which also lies generally parallel to the principal transverse axis (T) and runs from one longitudinal side 80 to the opposing longitudinal side 82. Thus, the pliable component 66 may have a width ranging between a minimum of no less than about 5 mm, up to a maximum of about 70 mm; although the approximate widths of the pliable member may vary according to, inter alia, the general design and intended disposition of the feminine care article within the vestibule 42 of a female wearer. One of skill in the art will readily appreciate that certain versions of the pliable component 66, and thus certain versions of the feminine care article, may have a minimum width (Wₘᵢₙ) equal to its maximum width (Wₘₐₓ). In such instances, reference is generally made only to the maximum width (Wₘₐₓ).

The pliable component 66 can have a maximum length (Lₘₐₓ), measured along a line laying generally parallel to the principal longitudinal axis (L) and running from one transverse end area to the other transverse end area 76, 78. Thus, the pliable component 66 may have a length ranging between no less than about 40 mm up to no greater than about 120 mm; although the approximate lengths of the pliable member 66 may vary according to, inter alia, the general design and intended disposition of the feminine care article within the vestibule 42 of a female wearer. One of skill in the art will readily appreciate that certain versions of the pliable component 66, and thus certain versions of the feminine care article, may have a minimum length (Lₘᵢₙ) equal to its maximum length (Lₘₐₓ). In such instances, as illustrated at least in FIG. 4, reference is generally made only to the maximum length (Lₘₐₓ). Versions of a pliable component 66, and thus versions of a feminine care article, having a maximum length (Lₘₐₓ) not equal to its minimum length (Lₘᵢₙ) are illustrated at least in FIGS. 7 through 10 and FIG. 15.

Similar to the pliable component 66, the feminine care article 40 can have a maximum width of up to about 70 mm, and can have a maximum length of up to about 120 mm. Additionally, the feminine care article can have a minimum width of not less than about 5 mm, and a minimum length of not less than about 60 mm.

The first end region 70 and the second end region 72 each minimally extend outwardly from the central region 74 toward the transverse end areas 76 and 78, respectively of the pliable component 66 a distance of no less than about 30%; alternatively, no less than about 20%; or alternatively, no less than about 10% of the maximum length (Lₘₐₓ) of the pliable component. The first end region 70 and the second end region 72 each maximally extend outwardly from the central region 74 toward the transverse end areas 76 and 78, respectively of the pliable component 66 a distance of no greater than about 20%; alternatively, no greater than about 30%; or alternatively, no greater than about 40% of the maximum length (Lₘₐₓ) of the pliable component. Thus, the end regions 70, 72 may occupy from a minimum of about 20% up to a maximum of about 80% of the maximum length (Lₘₐₓ) of the pliable component 66; although the approximate size of the first and second end regions may vary according to, inter alia, the general design and intended disposition of the feminine care article within the vestibule 42 of a female wearer.

The feminine care article (such as provided by the interlabial device 40) may optionally be configured with sufficient capacity to absorb and retain an intended amount and type of bodily fluids or exudates. The absorbent capacity may be provided by the pliable member 66, or may be provided by a liquid-retentive member or absorbent core. The absorbent core may be a part of the pliable member 66, or may be a separately provided component that is additional to the pliable member. In a particular feature, the absorbent can be a closely-formed structure or a relatively closed structure with a relatively small pore structure. For a selected bodily fluid, such as urine or menstrual fluid, the pliable component 66 or other absorbent member may have a minimum absorbent capacity of no less than about 1 gram of liquid (e.g. menses simulant) per gram of the absorbent (1 g/g), and may have a maximum capacity of about 30 g/g or more; although the approximate capacity of the absorbent may vary according to, inter alia, the general design and intended disposition of the feminine care article within the vestibule 42 of a female wearer. One of skill in the art will readily realize that superabsorbent polymers or coated superabsorbent polymers can be added to the pliable component 66 to thereby substantially increasing the absorbent capacity.

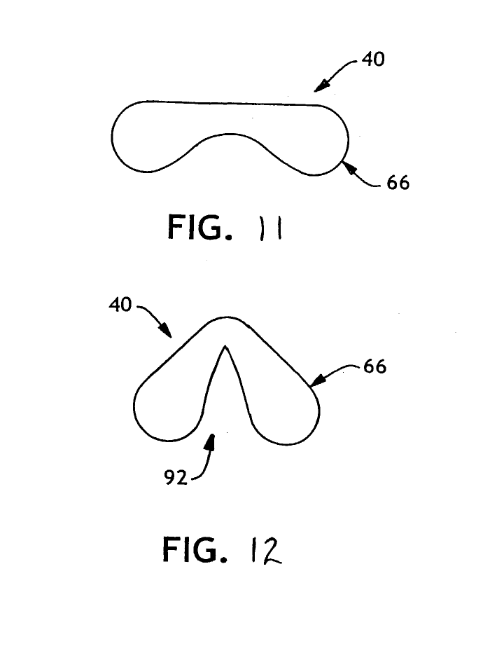

The pliable component 66 has an upper or body-facing surface and a lower surface or surface opposed to the upper or body-facing surface and may include a material capable of absorbing and/or adsorbing and thereafter retaining one or more bodily exudates. Suitable materials can also be generally hydrophilic, compressible, moldable and/or conformable. The pliable component 66 may be formed from any of the materials well known to those of ordinary skill in the art. Examples of such materials include, but are not limited to, various natural or synthetic fibers, multiple plies of creped cellulose wadding, fluffed cellulose fibers, rayon or other regenerated cellulose materials, wood pulp fibers or comminuted wood pulp fibers, airlaid material, textile fibers, a blend of polyester and polypropylene fibers, absorbent foams, absorbent sponges, superabsorbent polymers, coated superabsorbent polymers, fibrous bundles or nits, or any equivalent material or combination of materials. Also suitable for use would be hydrophobic material that has been rendered hydrophilic according to any of a number of known methods for so doing. The total absorbent capacity of the pliable component 66 should, however, be compatible with the design exudate loading and the intended use of the feminine care article. Further, the size and absorbent capacity of the pliable component 66 may be varied. Therefore, the dimension, shape, and configuration of the pliable component 66 may be varied e.g., the absorbent may have a varying thickness, as illustrated at least in FIGS. 11 and 12, or may have a hydrophilic gradient, or may contain superabsorbent polymers and the like.

The pliable component 66 generally has a thickness, caliper or height H, as illustrated at least in FIG. 5, measured along a line lying generally parallel to the z-axis. The minimum thickness of the pliable component 66 typically is no less than about 0.5 mm. Additionally, the pliable component 66 may have a maximum thickness of up to about 10 mm or more; although the approximate thickness of the absorbent may vary according to, inter alia, the general design and intended disposition of the feminine care article within the vestibule 42 of a female wearer. The caliper of a material is measured at a pressure of 0.05 psi (3.5 g/cm²), and can be measured in units of length (e.g. millimeters) with a STARRETT bulk tester, which is available from LS Starrett Company LTD, a business located in Jedburg, Scotland. Alternatively, a substantially equivalent testing apparatus may be employed.

The pliable component 66 can also have a relatively low density to help provide improved comfort. Generally, the absorbent can have a maximum density of not more than about 0.5 g/cm³. The pliable component 66 can also has a minimum density of no less than about 0.01 g/cm³. Additionally, the approximate density of the absorbent may vary according to, inter alia, the general design and intended disposition of the feminine care article within the vestibule 42 of a female wearer. In desired arrangements, the density of the pliable component may be at least about 0.05 g/cm³, and/or may be not more than about 0.2 g/cm³ to provide improved performance.

The pliable component 66 can have a maximum basis weight of up to about 600 grams per square meter (gsm). The pliable component 66 can also have a minimum basis weight of no less than about 0.1 g/m². Additionally, the approximate basis weight of the absorbent may vary according to, inter alia, the general design and intended disposition of the feminine care article within the vestibule 42 of a female wearer. A particular example of a suitable absorbent would be similar to a coform material made of a blend of polypropylene and cellulose fibers which has been employed in KOTEX brand, pantiliners and are obtainable from Kimberly-Clark Corporation, Neenah, Wis., U.S.A.

Various materials may be employed to form the pliable component or member 66. In desired configurations, the materials are operatively moldable and shapeable. Such materials can include, for example, open-cell or closed-cell foam materials, sponge materials, fibrous materials, gel materials, absorbent particles, adsorbent particles or the like, as well as combinations thereof.

The optional backsheet layer or baffle 64 typically resides on the lower surface of the pliable component 66, and may be constructed from any desired material. In a particular arrangement the backsheet or baffle layer 64 may be configured to be operatively liquid-impermeable. Desirably, the baffle 64 can be configured to be breathable. Accordingly, the baffle can permit the passage of air and moisture vapor out of the pliable component 66, while blocking the passage of bodily liquids. The baffle 64 may additionally be configured to be substantially biodegradable. An example of a suitable baffle material is a micro-embossed, polymeric film, such as polyethylene, polypropylene or polyester, having a minimum thickness of no less than about 0.025 mm and a maximum thickness of no greater than about 0.13 mm. Bicomponent films can also be used, as well as woven and nonwoven fabrics which have been treated to render them liquid-impermeable. An example of another suitable material is a closed cell polyolefin foam. A closed cell polyethylene foam may also work well.

The baffle 64 may be maintained in secured relation with the pliable component 66 by bonding all or a portion of the adjacent surfaces to one another. A variety of bonding methods known to one of skill in the art may be utilized to achieve any such secured relation. Examples of such methods can include, but are not limited to, ultrasonics, thermal bonding, or the application of adhesives in a variety of patterns between the two adjoining surfaces. A specific example of a baffle material would be similar to a polyethylene film used on KOTEX brand pantiliners and obtainable from Pliant Corporation, Schaumburg, Ill., U.S.A.

The optional topsheet or layer cover 62 has an upper surface and a lower surface, with the upper surface typically configured to contact the body of the wearer and to receive bodily exudates. Additionally, the cover may be configured to be operatively liquid-permeable. The cover 62 desirably is made of a material that is flexible and non-irritating to the tissues within the vestibule 42 of a female wearer. As used herein, the term “flexible” is intended to refer to materials which are compliant and readily conform to the bodily surfaces or respond by easily deforming in the presence of external forces.

The cover 62 can be configured to provide conformability and comfort to the wearer, and may optionally be configured to direct bodily exudates away from the wearer's body and toward the pliable component 66. The cover 62 should desirably retain little or no liquid in its structure, is desirably configured to provide a relatively comfortable and non-irritating surface next to the body tissues within the vestibule 42 of a female wearer. The cover 62 can be constructed of any operative material, such as provided by a film material, woven material, nonwoven material or the like, as well combinations thereof. Additionally, the cover can be configured to be easily penetrated by bodily liquids that contact the surface of the cover. Examples of suitable materials include rayon fabric, bonded carded webs of polyester, polypropylene, polyethylene, nylon, or other heat-bondable fibers, polyolefin materials, such as copolymers of polypropylene and polyethylene, linear low-density polyethylene, aliphatic esters such as polylactic acid, polymer films, finely perforated film webs, net material or the like, as well as combinations thereof. Other examples of suitable materials are composite materials of a polymer and a nonwoven fabric material. The composite materials are typically in the form of integral sheets generally formed by the extrusion of a polymer onto a web of spunbond material. The liquid-permeable cover 62 can optionally contain a plurality of apertures (not shown) that are formed partially or completed through the z-directional thickness of the cover layer. The apertures can, for example, increase the rate at which bodily fluids penetrate into the pliable component 66.

A physiologically hydrous cover material may also be employed. As used herein, the term “physiologically hydrous” is intended to connote a cover material which maintains a suitably moist interface between the tissues of the vestibule 42 and the feminine care article when disposed in that vestibular environment; one that is benign respecting the requirements of comfort associated with the interposition of fabric or fabric-like structures within the moist body-tissue environment of the vestibule, keeping in mind the fact that the feminine care article may be receiving bodily fluids migrating through the vestibule and may be arranged to conduct such bodily fluids to the pliable component 66 or other employed absorbent member. Thus, while not “hydrous” in the classic sense prior to use inasmuch as the cover will be dry at that time the cover 62 maintains or at least does not interfere with the maintenance of the proper moisture level or balance required within the vestibule 42.

The cover 62 may optionally include a treatment to selectively tailor the desired properties of the cover. The treatment may specifically aid in providing or maintaining a normal environment of the labia or aid in increasing the moisturization of the labia region. The treatment may or may not contain a skin care active ingredient. The formulation may, for example, help to reduce friction during placement or reduce drying of the inside surface of the wearer's labia. Examples of suitable cover materials are disclosed in U.S. Pat. No. 4,846,824 entitled LABIAL SANITARY PAD by F. Lassen et al. which was granted Jul. 11, 1989 (attorney docket No. 6657.4), the entire disclosure of which is incorporated herein by reference in a manner that is consistent herewith.

Optionally, the cover 62 may have at least a selected portion of its surface area treated with a surfactant to render the treated portion more hydrophilic. This can permit the incoming bodily fluids to more readily penetrate the cover 62, particularly the treated areas of the cover. The surfactant may also diminish the likelihood that the incoming bodily fluids, such as menstrual fluid, will flow off the cover 62 rather than being absorbed by the pliable component 66. The surfactant can be irregularly or discontinuously applied to the surface of the cover 62 that contacts or otherwise overlays the upper, bodyside surface of the pliable component 66. In a desired configuration, the surfactant can be applied to the relatively more-hydrophilic layer 28 of the cover. The surfactant can alternatively be substantially evenly distributed across at least a portion of the surface of the cover that contacts or otherwise overlays the upper surface of the pliable component.

A conventional hydrophilic cover or liner component positioned over an absorbent core or other absorbent member, however, can in many cases allow liquid to move upwardly from the core toward the wearer again and “rewet” the skin of the wearer. It can also allow liquid to spread from the target area to the sides of the pad so that the stained area is much larger than that, for example, exhibited by a film covered pad. These are regarded as significant negative factors in the design of disposable personal care products since they can result in staining of clothing and bedding, and discomfort to the wearer.

To address this situation, conventional absorbent articles have included a hydrophobic layer placed below the hydrophilic liner to reduce the ability of liquid to move upwardly from the wetted core. For example, conventional structures have included a fibrous liner wherein a hydrophilic apertured nonwoven layer has been laminated with a hydrophobic apertured nonwoven layer with a hydroentanglement process. Additionally, the hydrophilic nonwoven layer has been positioned on a bodyside of the article, and the hydrophobic nonwoven layer has been positioned between said hydrophilic nonwoven layer and an absorbent. For example, see U.S. patent application Ser. No. 09/990,787 entitled COVER SHEET FOR PERSONAL CARE PRODUCTS filed Nov. 14, 2001 (attorney docket No. 16,791), which corresponds to PCT application PCT/US02/13530, filed Apr. 30, 2002. Also see PCT patent publication WO 00/71067 entitled FLUID INTAKE INTENSIFIER which was published Nov. 30, 2000.

In contrast to such conventional structures, the inventors have found that it is advantageous to configure a first, relatively more-hydrophilic, layer-portion or stratum 28 as a garment-facing, absorbent-side of the cover component. Additionally, it has been advantageous to position a second, relatively less-hydrophilic, layer-portion or stratum 30 as the bodyside part of the cover component 62 that is appointed for placement in contact with the wearer. Accordingly, the more-hydrophilic layer portion 28 can be positioned and held between the second layer portion 30 and the absorbent member 66. This configuration can further increase the rate of absorption of liquid.

Placing a relatively more-hydrophilic layer portion 28 of the cover 62 towards the absorbent member 66 can help remove liquid from the bodyside of the cover and direct the liquid into the absorbent member. Additionally, the relatively more-hydrophilic layer portion can help to more efficiently distribute liquid, taking the liquid away from the target area of the article and distributing the liquid along the absorbent member. The relatively more-hydrophilic layer portion 28 of the cover 62 can also provide a more effective void volume within which the incoming liquid can be temporarily stored prior to be absorbed by the absorbent member.

The cover 62 may comprise a substantially unitary nonwoven fabric having a first major facing-surface portion and an opposed, second major facing-surface portion. The hydrophilic, first layer portion 28 of the cover can be provided by the first facing-surface portion of the nonwoven fabric; and the relatively less-hydrophilic, second layer portion 30 of the cover can be provided by the second facing-surface portion of the fabric. In an additional feature, the hydrophilic, first layer portion can include a surfactant. For example, the cover 62 may be nonwoven fabric which includes a bonded-carded-web (BCW) made of polypropylene and polyethylene that has been used as a cover stock for KOTEX brand pantiliners and has been obtained from Sandler Corporation, Germany. An operative amount of a surfactant (e.g. CETIOL 1414E surfactant; available from COGNIS CORPORATION, a business having offices located in Cincinnati, Ohio, U.S.A.) may be applied to the appointed first facing-surface of the nonwoven fabric to thereby provide the desired relatively more-hydrophilic layer portion 28 of the cover.

The cover 62 may alternatively comprise a first nonwoven fabric layer, and a second nonwoven fabric layer. The first nonwoven layer can be configured to provide the hydrophilic first layer portion 28 of the cover; and the second nonwoven layer can be configured to provide the relatively less-hydrophilic second layer portion 30 of the cover. Accordingly, the second nonwoven layer can be positioned on a bodyside of the article, and the first nonwoven layer can be positioned between said second nonwoven layer and the selected absorbent member.

The cover 62 may optionally comprise a nonwoven layer which has been operatively joined to a polymer film layer, or a first polymer film layer which has been operatively joined with a second polymer film layer. The appointed bodyside layer of the cover can be operatively configured to be relatively less-hydrophilic, and the appointed absorbent-side layer of the cover can be operatively configured to be relatively more-hydrophilic.

Still another configuration can include a cover 62 wherein a first apertured nonwoven fabric layer has been configured to provide the hydrophilic first layer portion 28 of the cover; and a second apertured nonwoven layer has been configured to provide the relatively less-hydrophilic second layer portion 30 of the cover. Accordingly, the second apertured nonwoven layer can be positioned on a bodyside of said article; and the first apertured nonwoven layer can be positioned between said second nonwoven layer and the selected absorbent member.

Where the cover 62 includes two or more separately provided layers, the layers may be joined by any operative technique, such as chemical bonding, thermal bonding or the like. A desired arrangement, however, can include a cover 62 wherein the first nonwoven fabric layer and the second nonwoven fabric layer have been laminated or otherwise joined with a process which hydroentangles the first nonwoven fabric with the second nonwoven fabric. Accordingly, the first and second nonwoven layers may be operatively joined to form a composite by using a laminating process which involves no chemical bonding or thermal bonding operations.

In particular aspects, either or both of the first and second layers (28, 30) of the cover 62 can be apertured. An aperturing of the less-hydrophilic layer 30 can address the problem posed by the less-hydrophilic layer's tendency to impede liquid passage. An aperturing of the relatively more-hydrophilic layer 28 can provide a rapid, open pathway to the absorbent core for liquid arriving from the bodyside surface of the cover 62. Once liquid passes through the apertures, the liquid can spread out below the hydrophobic layer and go into an absorbent core (e.g. an absorbent pliable member 66). Since the apertures are a small percentage of the surface area of the multi-layer cover 62, the amount of liquid going back upward through them is significantly smaller than the amount of liquid that can pass upwardly through a conventional hydrophilic liner.

Aperturing of the component layers of the laminate may occur after, during or before a hydroentangling of the fibers in the cover member 62, which is discussed below. The apertures in the first layer 28 of the cover may or may not be aligned with the apertures in the second layer 30. Desirably, the laminate is apertured after the fibers in the cover member have been hydroentangled. Aperturing may be carried out by any means known in the art, including mechanical pin aperturing, die cutting, or forming the materials in such a way that they are produced with holes in place. The apertures may also be made through the use of high pressure water jets, which may occur while the fabrics are being hydroentangled. The surface area of the liner may be apertured to produce from between 10 and 50 percent open area, more particularly between 20 and 40 percent, and still more particularly about 25 percent.

The use of the hydroentangling process to join the layers, instead of chemical or thermal bonding means, can produce a composite laminate which is substantially free of melted fiber cross-over points. This avoids the production of relatively large masses of thermoplastic that can impede fluid movement. High pressure water entangling may also be used to remove a non-durable hydrophilic surface treatment from the hydrophobic layer during processing.

The relatively more-hydrophilic layer 28 may include naturally hydrophilic fibers such as cotton and rayon, or may include synthetic fibers that are naturally hydrophobic but have been treated to render the fibers hydrophilic. If the fibers are synthetic fibers that have been treated to be hydrophilic, the treatment must be sufficiently durable to withstand the rigors of hydroentangling. It is not required that all of the fibers of this layer be hydrophilic, just that the layer be predominately hydrophilic. The layer may be made from a blend of fibers.

The fibers from which a second, less-hydrophilic stratum or layer 30 may be made can include naturally hydrophobic fibers, such as synthetic polymer fibers. It is not required that this second stratum or layer be hydrophobic, just that the second layer be relatively less-hydrophilic. More particularly, the second stratum or layer is desirably, relatively less-hydrophilic than the first stratum or layer. The second layer may be made from a single type of fiber, or from a blend of fibers. As mentioned above, hydroentangling operation can also be used to remove a previously applied non-durable hydrophilic surface treatment from the hydrophobic layer during processing, thus rendering it hydrophobic again.

The fibrous nonwoven strata or layers that can be employed with the present invention may be made from any nonwoven process know in the art, including airlaying, spunbonding, meltblowing and carding of staple fibers. Each of the nonwoven layers may have an individual-layer, basis weight within the range of from about 0.25 ounce per square yard (osy) to about 3 osy (about 8.5 g/m² to about 102 g/m²).

Synthetic fibers include those made from polyolefins, polyamides, polyesters, acrylics, LYOCELL regenerated cellulose, Lenzing's viscose rayon, and any other suitable hydrophobic synthetic fibers known to those skilled in the art. Many polyolefins are available for fiber production, for example polyethylenes such as Dow Chemical's ASPUN® 6811A linear-low-density-polyethylene, 2553 LLDPE and 25355 and 12350 high density polyethylene are such suitable polymers. The polyethylenes have melt flow rates, respectively, of about 26, 40, 25 and 12. Fiber forming polypropylenes include Kolon Glotec's T-1001, Exxon Chemical Company's ESCORENE PD 3445 and Montell Chemical Co.'s PF304. Other polyolefins are also available. Fibers having a lower melting polymer component, like conjugate and biconstituent fibers are suitable for use as well. Such fibers include conjugate fibers of polyolefins, polyamides and polyesters like the sheath core conjugate fibers available from KoSa Inc. (Charlotte, N.C.) under the designation T-255 and T-256.

Natural fibers include wool, cotton, flax, hemp and wood pulp. Wood pulps include standard softwood fluffing grade such as CR-1654 (US Alliance Pulp Mills, Coosa, Ala.). Pulp may be modified in order to enhance the inherent characteristics of the fibers and their processability.

The relatively more-hydrophilic layer is desirably made from hydrophilic fibers, and may include a blend of hydrophilic fibers with a minor amount of hydrophobic fibers. The hydrophilic fibers should be present in an amount from about 50 to 100 percent, more particularly from 70 to 100 weight percent and still more particularly 80-100 weight percent.

The relatively less-hydrophilic layer predominately has fibers which are hydrophobic or relatively less-hydrophilic. The low cost of polypropylene fibers makes it an excellent choice for such a product and polypropylene fibers in an amount of as much as 100 weight percent may be used. Blends of polypropylene with other fibers like PET can also function well.

In a particular feature, the cover 62 can include a spunlace material. The spunlace cover material can include a mechanically bonded, nonwoven fabric in which fibers of a fibrous web have been entangled with the aid of thin jets of air or liquid to provide an interlocking of the fibers and the fiber structure. This process is often referred to as an entangling process. If water is used as the processing liquid, then the process is referred to as hydroentangling. The entangled material has pronounced textile-like properties, in comparison with other nonwoven fabrics. Additionally, the properties of the entangled material can be readily varied by appropriately selecting the types of fibers, the fiber mixtures, the degree of entanglement, the structure of the substrate employed to support the fibrous web during the entangling operation, etc.

For the purposes of the present invention, the fiber mixture and method of manufacturing are chosen such that a hydroapertured, hydroentangled spunlace cover material is produced in which one side of the cover material predominantly comprises fibers that are hydrophilic in nature while the other side of the cover material predominantly comprises fibers that are less hydrophilic in nature. Therefore, one side of the cover material will be more hydrophilic than the other side of the cover material. Contact angle measurements with water can be used to determine the hydrophilic nature of a material. “Hydrophilic” materials generally form contact angles with water of 90 degrees or less, while “hydrophobic” materials generally form contact angles with water of greater than 90 degrees. When comparing materials, those that form larger contact angles with water are less hydrophilic than those that form smaller contact angles with water.

Because of the small size of a labial or miniform article, as well as its orientation during wear, it is important that it utilizes as much of its absorbent capacity as possible. During wear, the miniform is worn folded with the tabs facing down rather than flat. Therefore, gravitational forces will work to cause premature leakage if the intake and distribution functions of the miniform do not perform effectively. Because of the presence of rayon fibers on the side with the PET/PP fibers as well as the apertures that allow access to the rayon layer underneath it, fast intake of fluid can still be achieved if the cover is oriented on an absorbent article such that the PET/PP layer faces the body of the wearer. The advantage of orienting the cover so that the relatively more-hydrophilic side (e.g. rayon side) faces the absorbent core of the miniform is improved fluid distribution through the absorbent core of the product. Having the relatively more-hydrophilic side (e.g. rayon side) side facing the absorbent core can allow for a better interaction between the cover and the absorbent. The rayon or other hydrophilic fiber can directly contact at least a portion of the absorbent member, and can also help create a continuous hydrophilic gradient from the body-side of the cover to the absorbent core. Therefore, the miniform is better able to draw fluid away from the top PET/PP layer of the cover, wick along the rayon layer, and be absorbed by the cotton/rayon absorbent.

In a desired configuration, at least an operative portion of the cover 62 is configured and positioned in a substantially direct contact with a pliable, absorbent component 66. The cover 62 may be maintained in secured relation with the pliable component 66 by bonding all or a portion of the adjacent surfaces to one another. A variety of bonding methods known to one of skill in the art may be utilized to achieve any such secured relation. Examples of such methods include, but are not limited to, the application of adhesives in a variety of patterns between the two adjoining surfaces, entangling at least portions of the adjacent surface of the absorbent with portions of the adjacent surface of the cover, or fusing at least portions of the adjacent surface of the cover to portions of the adjacent surface of the absorbent.

The cover 62 typically resides on the upper surface of the pliable component 66, but alternatively can surround and partially or entirely enclose the absorbent. Alternatively, the cover 62 and the baffle 64 can have peripheries which extend outwardly beyond the periphery of the pliable component 66 and can be peripherally joined together to form an outboard edge region 84, as illustrated at least in FIG. 6. Utilizing known techniques, such as, for example, gluing, crimping, hot-sealing or the like, the edge region 84 may be formed entirely, so that the entire periphery of the pliable component 66 is circumscribed by their joinder. Alternatively, the cover 62 and the baffle 64 can be partially peripherally joined along selected portions of the entire periphery. To minimize the possibility of irritation and/or discomfort to the wearer of the feminine care article, it is desired that the edge region 84 and at least the area of the feminine care article immediately adjacent the edge be soft, compressible and conformable. Desirably, the edge region 84 can have a minimum width of no less than about 0.5 mm, and can have a maximum width of up to about 10 mm. Additionally, the approximate width of any edge portion may vary according to, inter alia, the general design and intended disposition of the feminine care article within the vestibule 42 of a female wearer. In other optional embodiments, the cover 62 and/or the baffle 64 can have a periphery that is substantially coterminous with the periphery of the pliable component 66.

Positioned either on or substantially parallel to the principal longitudinal axis (L) of the pliable component 66, there may optionally be a desired axis of flexure. A desired axis of flexure generally runs in the longitudinal direction, i.e., along the x-direction, and may be off center from the principal longitudinal axis (L) by a selected distance. Desirably, the desired axis of flexure is aligned along the principal longitudinal axis (L). Additionally, the axis of flexure can extend about 40-100% of the maximum length (Lₘₐₓ) of the pliable component 66. A desired axis of flexure may result naturally from the dimensions, shape, and/or configuration of the pliable component 66, or the pliable component may be imparted with a weakened axis or region to create a desired axis of flexure. A desired axis of flexure may also be formed by any of the techniques known to one of skill in the art, including, for example, scoring, pre-folding, slitting, embossing, or the like. Although a desired axis of flexure is described herein as residing in the pliable component 66, one of skill in the art will readily appreciate that a desired axis of flexure may be formed in the cover 62, the baffle 64 and/or the absorbent; the cover and the baffle; the cover and the absorbent; or the baffle and the absorbent. When present, a desired axis of flexure typically allows a feminine care article (e.g. the interlabial device 40) to be folded more easily prior to disposition within the vestibule 42 of a female wearer.

The feminine care article also has a thickness, caliper or height H, as illustrated at least in FIGS. 5 and 6, measured along a line laying generally parallel to the z-axis. The minimum thickness of the feminine care article can be not less than about 0.5 mm; and the maximum thickness can be up to about 10 mm; although the approximate thickness of the feminine care article may vary according to, inter alia, the general design and intended disposition of the feminine care article within the vestibule 42 of a female wearer.

The feminine care article (e.g. the interlabial device 40) can optionally be configured to be folded along an axis lying on or positioned parallel to the principal longitudinal axis (L), as illustrated at least in FIGS. 12, 13 and 14, prior to disposition within the vestibule 42 of the female wearer. When folded along such an axis, the feminine care article will form a recess 92 which protects the wearer's fingers from soiling when the feminine care article is disposed within the vestibule 42. Once inserted, the feminine care article may have a tendency to unfold in an attempt to fill the vestibule and thus maintain the upper surface of the feminine care article in contact with the body tissues of the vestibule 42. The feminine care article may be resiliently biased along the axis about which it is folded to increase the tendency of the feminine care article to unfold. Alternatively, the pliable component 66 of the feminine care article may be thicker along its longitudinal edges, as illustrated at least in FIGS. 12 and 13, thus also demonstrating a biasing effect, if desired, which is typically intended to allow the upper surface of the feminine care article to contact the tissues of the vestibule 42. A feminine care article as described herein, however, does not necessarily require any additional features to maintain contact with the body tissues of the vestibule 42 of the female wearer. The naturally moist surfaces of the tissues of the vestibule 42 typically demonstrate a tendency to maintain contact with the upper surface of the feminine care article.

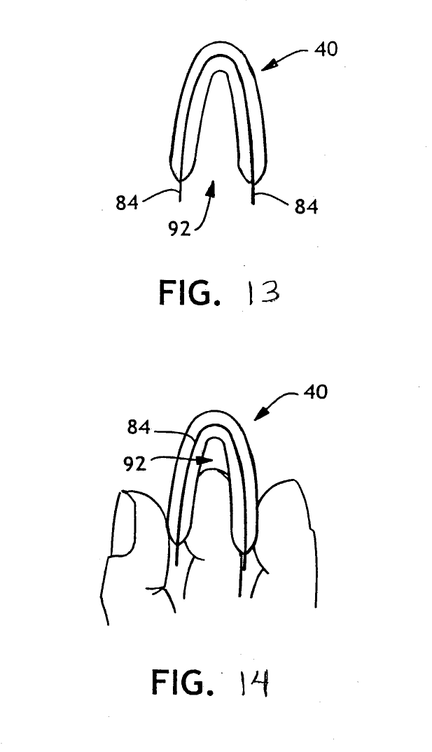

As noted above, the wearer may fold the feminine care article along an axis lying on or positioned parallel to the principal longitudinal axis (L) prior to disposition within the vestibule 42. The wearer may, therefore, hold the folded feminine care article at the longitudinal sides as illustrated at least in FIG. 14. The feminine care article may then be disposed within the vestibule 42 by the wearer exerting a force with a finger or fingers positioned in the recess 92 formed by the folded feminine care article.

As illustrated at least in FIGS. 3, 7 through 10 and 15, the pliable component 66, and thus the feminine care article, may be provided with at least one notch 100 extending inward from the periphery. As used herein, the term “notch” refers to a space, indentation or hollow region along the periphery of a material, a layer of material, a laminate of materials or other composite of materials. Because of the numerous possible geometries for the pliable component 66, and thus the feminine care article, it is almost impossible to indicate where on a particularly configured feminine care article the notch 100 should be located without seeing that particular feminine care article in use. However, it has been determined that when located at least in the periphery of that portion of the feminine care article that is to be situated nearest the clitoris 60, the notch 100 can help maximize the possibility that the feminine care article will maintain a substantially spaced relationship from a female wearer's clitoris when the feminine care article is disposed in a female wearer's vestibule 42. Such a spaced relationship can help minimize the likelihood that the feminine care article will contact the sensitive clitoris 60, thus guarding against the irritating and perhaps painful chafing effects which can arise from excessive contact between the clitoris and the feminine care article.

Additionally, the desired incorporation of the notch region in the feminine care article, and the desired placement of the notch region in a position close to the clitoris can allow the wearer to better control the use of the feminine care article. In a particular aspect, the notch region can be configured to substantially avoid placement over the wearer's urethra during ordinary and customary use. Thusly configured, the feminine care article can more effectively remain in position in the vestibule during and after urination. As a result, the wearer can have more control over when the feminine care article is removed and/or replaced. Additionally, the feminine care article will not be automatically or uncontrollably discharged from the vestibule during urination and require replacement. The increased control over the discharge of the feminine care article can render the article more convenient and more economical to use.

It has also been determined that when a notch region 100 is located at least in the periphery of that portion of the feminine care article to be situated nearest the perineum 50, the notch can help minimize the likelihood that the feminine care article will come into irritating contact with the sensitive perineal region. This is believed to be significant for those wearers who would use the feminine care article post-partum when the perineal region is highly sensitized or has been sewn due to tearing or having been cut during childbirth. It is noteworthy, however, that even those wearers who are nulliparous, i.e., the perineal region has not been exposed or experienced stretching, tearing or cutting during childbirth, may also have highly sensitive perineal regions.

The pliable component 66, and thus the feminine care article, may include at least one notch 100 extending inward from the periphery of at least one of the transverse end areas 76, 78. The notch 100 may, for example, be situated substantially on or adjacent to the principal longitudinal axis (L) of the pliable component 66. Alternatively, the notch 100 may be situated substantially on or adjacent to a desired axis of flexure. The notch 100 may also provide a natural folding or bending line to the pliable component 66 thus allowing the feminine care article, when folded or bent along any such line, to be more easily folded or bent. This is believed to be particularly true when a pliable component 66 (e.g. as illustrated at least in FIGS. 9, 10 and 15) has at least one notch 100 situated in the periphery of each opposing transverse end area 76, 78. As illustrated, the pliable component 66 and/or the article can have at least two notches 100 situated in the article periphery. For example, there can be one notch at or near opposing ends of an axis or line of the article (e.g. FIG. 16). When the feminine care article is disposed within the vestibule, and when the notch 100 is located at least in the periphery of the transverse end area that is to be situated nearest the clitoris 60, the configuration can help minimize the likelihood that the feminine care article will irritatingly contact the sensitive clitoris.

Although previously described herein as capable of being optionally folded along an axis lying on or positioned parallel to the principal longitudinal axis, the feminine care article (e.g. the interlabial device 40) may also be folded along an axis lying on or positioned parallel to the principal transverse axis (T) prior to disposition within the vestibule 42 of the female wearer. When folded along such an axis, the feminine care article typically still forms a recess 92 which protects the wearer's fingers from soiling when the feminine care article is disposed within the vestibule 42. Once inserted, the feminine care article may have a tendency to unfold in an attempt to fill the vestibule and thus maintain the upper surface of the feminine care article in contact with the tissues of the vestibule 42. The feminine care article may be resiliently biased along the axis about which it is folded to increase the tendency of the feminine care article to unfold. Alternatively, the pliable component 66 of the feminine care article may be thicker along its transverse end areas 76, 78 thus also demonstrating a biasing effect, if desired, which is typically intended to allow the upper surface of the feminine care article to contact the tissues of the vestibule 42. A feminine care article as described herein, however, does not necessarily require any additional features to maintain contact with the tissues of the vestibule 42 of the female wearer. The naturally moist surfaces of the tissues of the vestibule 42 typically demonstrate a tendency to maintain contact with the upper surface of the feminine care article.

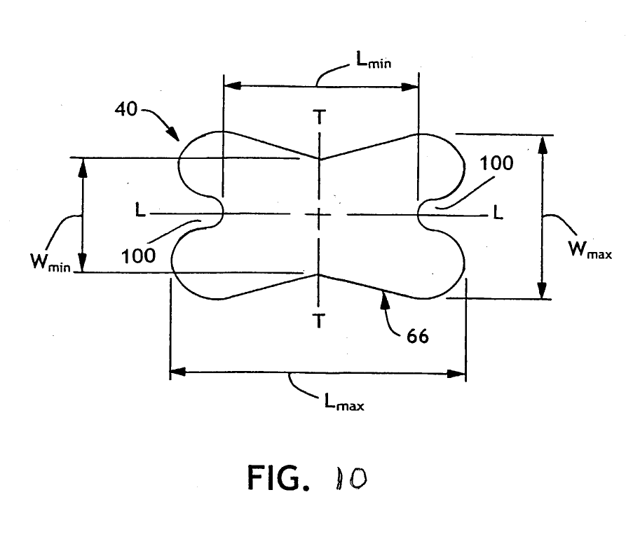

In another version, a pliable component 66, and thus a feminine care article, includes at least one notch 100 extending inward from the periphery of at least one of the longitudinal sides 80, 82. The notch 100 may, for example, be situated substantially on or adjacent to the principal transverse axis (T) of the pliable component 66. The notch 100 may also provide a natural folding or bending line to the pliable component 66 thus allowing the feminine care article, when folded or bent along any such transverse axis or line, to be more easily folded or bent. This is believed to be particularly true when a pliable component 66, a version of which is illustrated at least in FIG. 10, has at least one notch 100 situated in the periphery of each opposing longitudinal side 80, 82. When the feminine care article is disposed within the vestibule, the notch 100, when located at least in the periphery of the longitudinal side to be situated nearest the clitoris 60, minimizes the likelihood that the feminine care article will irritatingly contact the sensitive clitoris.

Desirably, the notch 100 is of dimensions sufficient to minimize the likelihood that the feminine care article will, when appropriately disposed within a female wearer's vestibule 42, come into irritating contact with the clitoris 60 and/or the perineum 50, as desired. Stated differently, the notch 100 desirably is of dimensions sufficient to maximize the possibility that the feminine care article will maintain a substantially spaced relationship from the clitoris 60 and/or the perineum 50, as desired, when the feminine care article is appropriately disposed within a female wearer's vestibule 42.

The notch 100 suitably extends inward from the periphery of the pliable component 66 by a depth, as measured approximately perpendicularly from the peripheral, terminal edge of the absorbent, For example, the notch 100 may have a minimum depth of no less than about 2 mm, and may have a maximum depth of up to about 30 mm; although the approximate depth of the notch may vary according to, inter alia, the general design and intended disposition of the feminine care article within the vestibule 42 of a female wearer.

The notch 100 also has a width, the widest portion of which is typically situated at least along the periphery of the pliable component 66. Desirably, the notch 100 has a maximum width of up to about 30 mm, and a minimum width of not than about 0.5 mm; although the approximate width of the notch may vary according to, inter alia, the general design and intended disposition of the feminine care article within the vestibule 42 of a female wearer.

The notch 100 when configured as described herein may have a variety of geometries including U-shaped, V-shaped, W-shaped, semi-circular or a variety of combinations thereof. Several examples of possible notch 100 geometries are illustrated in several of the Figures. One of skill in the art will recognize, however, that the notch geometries identified herein are non-limiting and are but a few examples of the many geometries that may be suitable for the notch 100 described herein.

The notch 100 of the present invention may be situated on the periphery of a pliable component 66, and thus the periphery of a feminine care article, having a variety of geometries. Examples of such geometries of the article and of the components of the article can include, but are not limited to, rectangular, ovoid-like, elliptical, trapezoidal, circular, semi-circular, triangular, square-shaped, teardrop-like, diamond-shaped, polygonal-shaped, butterfly, pear-shaped, heart-shaped, or the like, as was well as any operative combination thereof.

Suitable interlabial devices are also described in U.S. patent application Ser. No. 10/036,981 entitled LABIAL PAD HAVING A TAB by Heather A. Sorebo et al. which was filed Dec. 31, 2001 (attorney docket No. 17,692). The entire disclosure of this document is incorporated herein by reference in a manner that is consistent herewith.

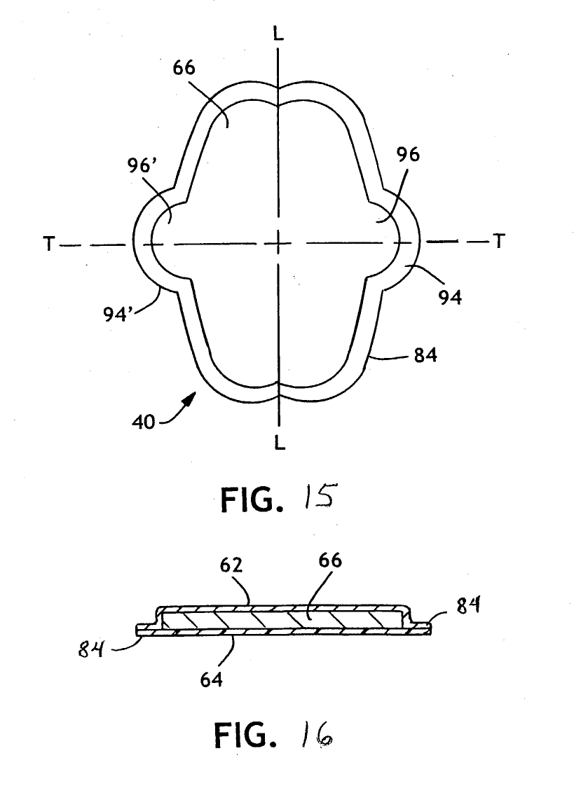

With reference to FIGS. 15 and 16, the feminine care article (e.g. such as provided by the interlabial device 40) can include at least one, placement and removal tab region 94 which extends outward from at least one longitudinal side 80, 82 of the feminine care article. Each tab region may or may not include a corresponding section of absorbent material 96, as desired. While one such tab 94 may conceptually work effectively in the placement and removal of a feminine care article such as a labial pad, it is believed that at least two tabs 94 and 94′, i.e., one tab extending from each longitudinal side 80, 82 of the feminine care article, can be more effective in the placement and removal of a feminine care article. Consequently, in the discussion that follows, unless otherwise noted, the feminine care article can have at least two tabs 94, 94′. While it is not necessary, the tabs 94, 94′ can be identical, or more properly, mirror images each other. Thus, the description of the first tab will be a corresponding description of any other tab. Discussion of any other tab will, therefore, be omitted for clarity of exposition. Corresponding elements are indicated in the drawings by reference numerals and primed reference numerals.

Extending outward from a longitudinal side 80, 82 of a feminine care article, the tab 94 can be of any suitable configuration. Non-limiting examples of shapes for the tab 94 include: ovoid, elliptical, trapezoidal, rectangular, triangular, diamond-shaped, circular, semi-circular, or the like, as well as any combination thereof. The tab 94 may be integrally formed with the feminine care article or it may be a separately provided element that is joined to the feminine care article. One of skill in the art will readily appreciate that when the tab 94 is a separate element joined to the feminine care article, the tab may be so joined by a number of known methods including melt fusion, adhesion, or other joining means. The phrase “integrally formed” is intended to indicate that the tab 94 is a continuous extension of the cover 62, the baffle 64, the pliable component 66 or a like component, as well as a continuous extension of a combination of such components.

The tab 94 has a length measured along a line laying generally parallel to the principal longitudinal axis (L) of a feminine care article, and a width, measured along a line laying generally parallel to the principal transverse axis (T) of a feminine care article. The tab 94 has sufficient dimensions to aid the female user in disposition of the feminine care article within the vestibule 42 and, optionally, removal of the feminine care article from the vestibule. The phrase “sufficient dimensions” is intended to indicate that the tab 94 can be grasped between the index finger and the thumb or, if there are, for example, two tabs, between the index finger and the thumb and the middle finger and the index finger. Typically, the length of the tab 94 is no greater than the maximum length (Lₘₐₓ) of the pliable component 66. One of skill in the art will readily appreciate that the length of the tab 94 may vary according to, inter alia, the general design and intended disposition of the feminine care article within the vestibule 42 of a female user.

In addition to having a length, the tab 94 also has a width. One of skill in the art will readily appreciate that the width of the tab 94 may vary according to, inter alia, the general design and intended disposition of the feminine care article within the vestibule 42 of a female user.

The dimensions of the tab 94 are limited only by the stress-strain properties of the tab materials. Desirably any material used in the tabs 94, 94′ is soft, compressible and conformable and thus similar to the material used in the fluid permeable cover 62, the liquid impermeable baffle 64 and/or the pliable component 66. Any such material is desirably configured to minimize the possibility of irritation and/or discomfort to the wearer of the feminine care article. Additionally, the tab material should have sufficient strength and integrity to allow a grasping of the tabs to place the article on the wearer.

The tab 94 may be positioned in a variety of locations along the longitudinal side 80, 82 of a feminine care article. With regard to the feminine care articles 40 described herein, the tab 94 may be located in the first end region 70, the second end region 72 or the central region 74. A second tab 94′ could at the same time be located along the opposing longitudinal side 80, 82 in the first end region 70, the second end region 72 or the central region 74. Generally, when a tab 94 extends outward from a longitudinal side 80, 82 of a particular region 70, 72, 74, any second tab 94′ typically extends outward from the corresponding region 70, 72, 74 of the opposing longitudinal side 80, 82. It should also be noted that, depending on the length of the tab 94, the tab may cover more than one of the regions 70, 72, 74 described herein. The tabs 94, 94′ as described herein offer a female wearer the opportunity to grasp the tabs to aid in the disposition of a labial pad into the vestibule. In addition, the tabs 94, 94′ also offer a female wearer the opportunity to grasp the tabs to aid in the removal of a labial pad and thus minimize the likelihood that the female wearer's fingers will come into contact with the body-facing surface of the possibly soiled labial pad.

Other suitable interlabial devices are described in U.S. patent application Ser. No. 10/038,973 entitled LABIAL PAD HAVING VARIOUS MEANS by James J. Hlaban et al. which was filed Dec. 31, 2001 (attorney docket No. 17,693). The entire disclosure of this document is incorporated herein by reference in a manner that is consistent herewith.

The pliable component 66 and the feminine care article may, for example, include a placement enhancement structure designed to minimize the surface area of that portion of the feminine care article that comes into contact with the floor 48 of the vestibule 42 when the feminine care article is disposed within the vestibule of a female wearer. Minimizing the surface area of that portion of the feminine care article that comes into contact with the floor 48 of the vestibule 42 is believed to guard against the irritating and perhaps painful chafing effects which contact by a feminine care article with the floor of the vestibule can occasion. For example, the placement enhancement structure can include at least one slit residing on an axis either lying on or running parallel and adjacent to the principal longitudinal axis (L). The placement enhancement structure may include a single continuous slit or a series of slits, and may extend a selected longitudinal distance along the length of the absorbent. For example, the longitudinal distance can have a minimum of no less than about 80% of the length of the pliable component 66, and a maximum of no greater than about 100% of the length of the pliable component 66. Alternatively, the placement enhancement structure can include at least one slit residing on an axis which either lies on or runs parallel and adjacent to the principal transverse axis (T). Accordingly, such a configuration can also include a single slit or a plurality of slits. When the selected configuration of the placement enhancement structure incorporates a series of slits, each slit can be spaced apart from an adjacent slit by a selected spacing distance. For example, the spacing distance can have a minimum of about 2 mm, and a maximum of about 15 mm.

The placement enhancement structure can also have a depth extending through the thickness of the pliable component 66. The approximate length, width, and/or depth of the placement enhancement structure may vary according to, inter alia, the general design and intended disposition of the feminine care article within the vestibule 42 of a female wearer.

In another configuration, the pliable component 66, and the feminine care article, may include a deformation structure which allows the feminine care article to substantially conform to the effective surface area of the vestibule 42 when the feminine care article is folded and disposed therein. The phrase “effective surface area” of the vestibule 42 is intended to refer to that portion of the vestibule that contacts the feminine care article. By substantially conforming to the effective surface area of the vestibule 42 when folded and disposed therein, the feminine care article is less likely to become dislodged away from the vestibule during use. In addition, by substantially conforming to the effective surface area of the vestibule 42, the feminine care article can help minimizes the likelihood of leakage. In a particular arrangement, the deformation structure can include at least one slit, and optionally, a selected plurality of slits, residing on an axis which either lies on or runs parallel and adjacent to the principal longitudinal axis (L). Whether a single continuous slit or a series of slits, the approximate length and/or depth of the deformation means may vary according to, inter alia, the general design and intended disposition of the feminine care article within the vestibule 42 of a female wearer. When incorporating a series of slits, each slit of the deformation structure can be spaced apart from an adjacent slit by a distance of about 2 mm to about 15 mm,

If the feminine care article is foldable, a female wearer may optionally fold the article along an axis which lies on or is positioned parallel to the principal transverse axis (T) prior to disposition within the vestibule 42. In such situations, the wearer may, therefore, hold the folded feminine care article at the transverse end areas when disposing the feminine care article within the vestibule 42. Taking into account such instances, the positioning of the deformation structure may be modified accordingly.

In a further arrangement, the pliable component 66, and the feminine care article may include a fluid intake enhancement structure. The fluid intake enhancement structure can contribute to increasing the surface area of the pliable component 66, and can be capable of allowing bodily fluids to be more rapidly absorbed into the pliable component 66, as compared to an identical feminine care article that does not contain such a fluid intake enhancement structure. The fluid intake enhancement structure can allow for rapid absorption of the intended bodily exudate without the typical fluid intake limitations that may be encountered when the upper surface of the pliable component 66 is substantially flat or concave, and is located adjacent the vaginal 56 or urethral 58 orifice. In a particular arrangement, the fluid intake enhancement structure can include at least one slit, and optionally, a plurality of slits, residing on an axis either lying on or running parallel and adjacent to the principal longitudinal axis (L).

Additional examples of interlabial devices are disclosed in U.S. patent application Ser. No. 10/037,276 entitled LABIAL PAD by Ronald L. Edens et al. which was filed Dec. 31, 2001 (attorney docket No. 17,696); and in U.S. patent application Ser. No. 10/038,971 entitled LABIAL PAD by Deanna R. Kathumbi-Jackson et al. which was filed Dec. 31, 2001 (attorney docket No. 17,697). The entire disclosure of this document is incorporated herein by reference in a manner that is consistent herewith.

In addition to considering the length and width of a labial pad or other interlabial device, the surface area of the labial pad or other interlabial device can also play a significant role in enhancing the comfort and fit of a labial pad disposed within a female wearer's vestibule. The effective surface area of the human female vestibule can be as small as about 275 mm², or even smaller depending on the female. In addition, the effective surface area of the human female vestibule can be as large as about 3,800 mm², or even larger depending on the female. Use of the phrase “effective surface area” with regard to a vestibule is intended to refer to that portion of the surface of the vestibule available for contact with feminine care articles similar to and including those described herein. Although there exists a great amount of variation in the effective surface area of the human female vestibule, a significant number of human female vestibules can have effective surface areas within the range of about 700 mm² to about 3,100 mm². By substantially matching the surface area of the upper surface of a pliable component 66 with the effective surface area of a female wearer's vestibule, the feminine care article can be configured to demonstrate an improved efficacy at maintaining a desired disposition within the vestibule 42. As a result, the feminine care article can provide better coverage of the vestibule, can better minimize the potential for leakage, and can provide enhanced comfort to the wearer. This is particularly significant when desiring to maintain a desired disposition of the feminine care article within the vestibule of a female wearer without the assistance of an additional stay-in-place mechanism, such as, for example, provided by strings, body adhesives, garment adhesives, belts, sanitary napkins, tampons, undergarments or the like.

The following Examples are presented to provided a more detailed understanding of the invention, and are not intended to specifically limit the scope of the invention.

### EXAMPLE 1

An absorbent feminine care article of this Example included a baffle, a cover, and a pliable absorbent core member that was sandwiched between the baffle and cover. The cover included a two layer laminate was made having a top or bodyside facing layer, and a bottom or absorbent-side layer. The bodyside, first nonwoven fabric layer was a 0.47 osy (16.5 g/m²) carded web having 73 weight percent PET and 27 weight percent polypropylene (PP) fibers. The absorbent-side, second nonwoven fabric layer was a 0.40 osy (13.5 g/m²) carded web and had 90 weight percent Rayon, naturally hydrophilic fiber and 10 weight percent polyethylene terephthalate (PET) fibers. The layers were hydroentangled at a water pressure of 435-725 psi (30-51 kgf/cm²) and apertured afterwards to provide an aperture-concentration of approximately 50 apertures per cm² by an operative processing at 580 psi (41 kgf/cm²). The apertures were approximately 0.06 mm in diameter or about 0.3 mm² in area. The apertures were roughly diamond shaped because the mesh upon which the laminate was supported was diamond shaped. Support media with other shapes would result in other shapes and sizes for the apertures. In the present invention, the cover material is distinctively arranged so that the relatively more-hydrophilic rayon layer is away from the wearer's body, and the relatively less-hydrophilic PET/PP layer is towards the body.

### EXAMPLE 2

An absorbent feminine care article of this Example included a baffle, a cover, and a pliable absorbent core member which was sandwiched between the baffle and cover. The article included the following:

| | | Supplier |
|---|---|---|
| | | Name and Address |
| Cover | 30-g/m² BAIKSAN Spunlace | Baiksan Co., Ltd. |
| | YK30H5 | 1234, Jeongwang-dong, |
| | | Siheong-si, Kyunggi-do, |
| | | Korea |
| Absorbent | 60% BBA-14 Cotton; and 40% | BBA Nonwovens |
| | LENZING Code 8257 Rayon. | 100 Elm Street |
| | Medium-size miniform article | Walpole, Massachusetts, |
| | contains 0.75-1.0 grams of | U.S.A. |
| | absorbent. Long-size miniform | Acordis Kelheim GmbH |
| | article contains 0.9-1.3 grams | Regensberger Strasse 109 |
| | of absorbent. | 93309 Kelheim |
| | | Germany. |
| Baffle | PLIANT, XC3-999-1521.0. | Pliant Corporation |
| | White, Polyethlyene film | 1701 1st Avenue |
| | | Chippewa Falls, |
| | | Wisconsin, U.S.A. |
| Adhesive | NATIONAL STARCH, | National Starch and |
| | 34-5610. Adhesive applied at | Chemical Co. |
| | 5-9 g/m² add-on. | 10 Finderne Avenue |
| | | Bridgewater, New Jersey, |
| | | U.S.A. |
| | | 14351 Hwy 21 |
| | | Enoree, South Carolina, |
| | | U.S.A. |

In Example 2, the spunlace cover material was hydroentangled and apertured, and included rayon, polypropylene (PP), and poly(ethylene terephthalate) or PET. Two starting fibrous webs were used to make the fibrous layers in the cover material. The first fibrous web for providing the first nonwoven fabric layer was 100 percent rayon. The second fibrous web for providing the second nonwoven fabric layer included 72.7 percent PET and 27.3 percent PP. In the present invention, the cover material is distinctively arranged so that the relatively more-hydrophilic rayon layer is away from the wearer's body, and the relatively less-hydrophilic PET/PP layer is towards the body. The total basis weight of the final cover material was 30 g/m². The cover had 13.5 g/m² of Rayon (45% of the total cover), 12 g/m² of PET (40%, of the total cover), and 4.5 g/m² of PP (15% of the total cover).

After the hydroentangling and hydroaperturing operations are completed, the component layers became partially inter-mixed, but the rayon fibers of the first nonwoven layer were predominantly on one side of the cover material while the PET/PP fibers of the second nonwoven layer were predominantly on the other, opposite side of the cover material. The first-layer side with the predominant amount of rayon fibers (hereafter referred to as the “rayon side”) was relatively more hydrophilic than the second-layer side containing the predominant amount of relatively less-hydrophilic PP/PET fibers (hereafter referred to as the “PET/PP side”). The first, relatively more-hydrophilic layer of the two-layer semi-hydrophobic spunlace for this example was composed of 100% Rayon fiber. The second, relatively less-hydrophilic layer was composed of 72.7% PET fiber & 27.3% PP fiber.

With the cover configured to have the rayon layer (more-hydrophilic layer) facing toward towards the absorbent, the liquid-intake time was unexpectedly reduced, as illustrated in Table 1.

TABLE 1
Double Gush Testing With 2-mL Insults of Menses Simulant

| | Mean Intake | | Mean Intake | |
|---|---|---|---|---|
| | Time (sec) | | Time (sec) | |
| | 1st insult | STD | 2^{nd} insult | STD |
| 22 gsm Sandler BCW | 5.06 | .84 | 19.18 | 1.08 |
| Spunlace untreated | 2.99 | .03 | 16.56 | 1.10 |
| (rayon to absorbent) | | | | |
| Spunlace (rayon to body) | 2.67 | .06 | 16.31 | 1.70 |

STD = standard deviation.
Time between first and second insults was 9 seconds.

While not intending to be bound by any particular theory, it is believed that since rayon is a natural wicking agent, having the hydrophilic (rayon) layer facing away from the body and towards the absorbent core member 66 helps direct the fluid into the absorbent core. The absorbent core member 66 is comprised of 60:40 (cotton:rayon), and can establish a substantially continuous liquid-communication between the cover and absorbent fibers and other absorbent materials in the absorbent core, thereby improving the capillary action between the cover 62 and the absorbent member 66. With the hydrophilic layer away from the body and towards the absorbent member 66, the cover 62 can help increase the available void volume for the liquid because the rayon fibers can provide a pseudo-surge or temporary-storage area until the liquid can penetrate into the absorbent core member 66.

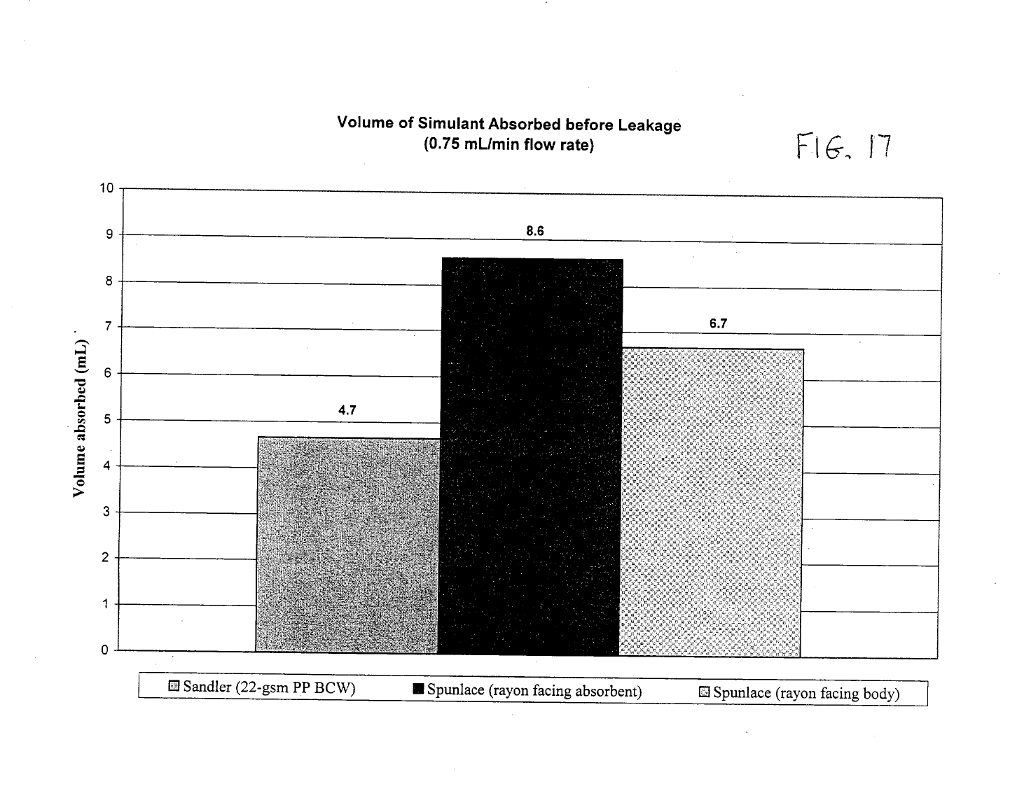

Bench tests with menses simulant have shown that a particular orientation of the spunlace cover with the rayon layer towards the absorbent core has resulted in a more effective utilization of the absorbent. The rate of liquid-intake has been improved. Additionally, a greater amount of liquid that can be absorbed prior to leakage, and stain sizes on the cover can be reduced. FIG. 17 provides a graphical overview of the liquid capacity comparison of interlabial devices with the various cover materials/orientations when tested with menses simulant. The product size, absorbent blend (cotton:rayon), and basis weight of the absorbent was the same for all miniform articles tested. The liquid-flow rate was 0.75 mL/minute, delivered from a tube 0.5 cm above the miniform article. The liquid-flow was stopped when the miniform article began to leak, and the total volume of liquid delivered at the leak point was recorded.

As can be seen from the graph of FIG. 17, a miniform article with the spunlace cover oriented with the rayon layer facing the body will not absorb as much liquid before leaking. Since the PET/PP layer is less hydrophilic than the rayon layer, some liquid can wick along the rayon layer before passing through the PET/PP layer and into the absorbent core. Eventually, the rayon layer will become saturated before the absorbent core, and liquid can run off the surface of the miniform article causing premature leakage.

In contrast, orienting the spunlace cover with the rayon layer facing away from the body will result in fast initial intake, and less leakage. With reference to FIG. 17, miniform articles with the cover oriented with the rayon layer facing the absorbent core were able to absorb more liquid than those with the rayon layer facing the body.

Dimensions of the stain size were also recorded for the codes tested with a Folded Miniform Intake and Distribution Test. Products were insulted with 3 mL of menses simulant at a flow rate of 0.75 ml/min. Stain sizes of the products were then measured. Results are given in Table 2.

TABLE 2
Stain Size Measurements After 3-mL Total Insult

| Cover Material Used | MD Stain Length | CD Stain Length |
|---|---|---|
| Sandler BCW 22 gsm | 1.3 cm | 3.4 cm |
| Spunlace (Rayon facing absorbent) | 1.3 cm | 1.6 cm |
| Spunlace (Rayon facing body) | 2.1 cm | 2.1 cm |

MD = along the primary longitudinal axis L
CD = along the primary transverse axis T

Miniform articles with a larger ratio of the CD-stain to the MD-stain tend to hold less fluid before leaking. The results for this CD-stain/MD-stain ratio correspond well to the results seen in FIG. 17.

Although the present invention has been illustrated and described in considerable detail with reference to certain embodiments thereof, other arrangements and configurations are also possible and are contemplated as being within the scope of the present invention. Therefore, the spirit and scope of the appended claims should not be limited to the specific illustrations and descriptions of the embodiments contained herein.

1. An absorbent feminine care article, comprising
a liquid-permeable cover; and an absorbent member operatively joined with said cover; wherein
said cover includes a hydrophilic, first layer portion operatively joined with a relatively less-hydrophilic, second layer portion;
said second layer portion is positioned on a bodyside of said article; and
said first layer portion is positioned between said second layer portion and said absorbent member.

2. An article as recited in claim 1 wherein
said hydrophilic first layer portion of the cover includes a first nonwoven layer;
said relatively less-hydrophilic second layer portion of the cover includes a second nonwoven layer;
said second nonwoven layer is positioned on a bodyside of said article; and
said first nonwoven layer is positioned between said second nonwoven layer and said absorbent member.

3. An article as recited in claim 1 wherein
said hydrophilic first layer portion of the cover includes a first apertured nonwoven layer;
said relatively less-hydrophilic second layer portion of the cover includes a second apertured nonwoven layer;
said second apertured nonwoven layer is positioned on a bodyside of said article; and
said first apertured nonwoven layer is positioned between said second nonwoven layer and said absorbent member.

4. An article as recited in claim 1 wherein
said cover comprises a nonwoven fabric having a first facing-surface portion and an opposed, second facing-surface portion;
said hydrophilic, first layer portion is provided by said first facing-surface portion; and
said relatively less-hydrophilic, second layer portion is provided by said second facing-surface portion.

5. An article as recited in claim 4 wherein said hydrophilic, first layer portion includes a surfactant.

6. An article as recited in claim 1 further including a baffle; and wherein said absorbent member is sandwiched between the cover and baffle;

7. An article as recited in claim 1 wherein said first layer portion includes apertures, said second layer includes apertures, and the apertures of said first layer portion are substantially aligned with the apertures of said second layer portion.

8. An article as recited in claim 1 wherein said first and second layer portions have been laminated according to a spunlace process.

9. An article as recited in claim 2 wherein the cover includes a first nonwoven layer comprising staple, naturally hydrophilic fibers hydroentangled to form a laminate with a second nonwoven layer comprising relatively less hydrophilic fibers, wherein said laminate is apertured with an aperture area of 10 to 50 percent.

10. An article as recited in claim 9 wherein said first layer comprises hydrophilic fibers selected from the group consisting of rayon, pulp, cotton, naturally hydrophilic fibers, and mixtures thereof.

11. An article as recited in claim 9 wherein said second layer comprises fibers made from polymers selected from the group consisting of polyolefins, polyesters, acrylics and mixtures thereof.

12. An article as recited in claim 1 wherein
said article is configured to provide a labial pad; and
said hydrophilic, first layer portion of the cover includes a first, hydrophilic apertured nonwoven layer;
said relatively less-hydrophilic second layer portion of the cover includes a relatively less hydrophilic, second apertured nonwoven layer; and
said first and second nonwoven layers have been laminated according to a spunlace process.
